(19) United States
(12) Patent Application Publication
(10) Pub. No.: US 2005/0124694 A1
(43) Pub. Date: Jun. 9, 2005

(54) Method of accelerating metabolism of alcohol

(76) Inventor: Arthur Vanmoor, Boca Raton, FL (US)

Correspondence Address: OTTO S KAUDER, 5382C Venetia Ct., Boynton Beach, FL, 33437, US

(21) Appl. No.: 10/731,802

(22) Filed: Dec. 9, 2003

(51) Int. Cl. A61K 31/205

(52) U.S. Cl. 514/554

(57) ABSTRACT

There is disclosed a method of assisting the elimination of alcohol in a person in need of such treatment, which comprises the administration to such person of an effective amount of at least one compound having the formula (I)
X—CH₂—CH(OY)_{q}(H)_{1-q}CH(NH₂⁺)_{1-q}COO⁻ (I)
and its addition salts, wherein X represents a trimethylammonio group (CH3)3N+ or a carbamoyl group (CONH₂), Y is hydrogen or an acetyl (CH₃CO) group, and q is zero or one, provided that when X is a carbamoyl group q is zero, and when X is a trimethylammonio group. q is one.

### BACKGROUND OF THE INVENTION

1. Field of the Invention

This invention relates to a method of treating a person seeking rapid relief and if possible prevention of the ill effects of ingesting alcohol including dizziness, nausea, blurred vision, lack of balance, and general impairment of skills such as the ability to speak clearly or to drive an automobile with an agent that enhances the catabolic effectiveness of the human liver to mitigate and where possible eliminate the after-effects of alcohol ingestion by accelerating the removal of alcohol from the person's circulatory system.

2. Description of Related Art

As is well known, remedies for treating the effects of alcohol have been sought for generations by a great variety of methods. However, the search by scientific techniques for better remedies for this as well as other suffering conditions is enormously costly. For economic reasons, moreover, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

In contrast, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds, are disclosed in U.S. Pat. No. 5,616,617 as effective against facial pimples; certain water soluble amino carboxylic acid compounds are disclosed in U.S. Pat. No. 5,626,831 as effective against the common cold; certain water soluble amino carboxylic acid compounds are disclosed in U.S. Pat. No. 5,707,967 as effective against headache; certain water soluble amino carboxylic acid compounds are disclosed in U.S. Pat. No. 5,708,029 as effective against elevated blood pressure, and certain water soluble amino carboxylic acid compounds are disclosed in U.S. Pat. No. 5,767,157 as effective against pain in a joint.

Vanmoor U.S. Pat. No. 6,476,073 discloses a method of treating ill effects of alcohol ingestion in a person in need of such treatment, which comprises the administration to such person of at least one aliphatic sulfur compound.

Published PCT application PCT/US 01/00714 discloses treatment of an ailment, which can be the effects of-alcohol consumption commonly known as “hangover”, with an aliphatic sulfur compound.

In recent years, concern has grown about the effects of alcohol consumption on others beside the one who consumes the alcohol, including the efforts to enforce laws against “DUI driving under the influence” of alcohol and to persuade pregnant women to avoid consuming alcohol. “Alcohol's Toll On Fetuses: Even Worse Than Thought”, an article by Linda Carroll published in the Science Times section of the New York Times for Nov. 4, 2003, notes that “It is not surprising that it has taken researchers so long to tease out the link between alcohol exposure and birth defects. For one thing, the effects of alcohol exposure seem to vary widely. Some fetuses seem to escape unscathed, even when their mothers drink heavily, while others are severely damaged. No one knows why.”

One result of the increased concern about both short term and long term effects of alcohol consumption has been the passage of laws limiting the amount of alcohol drivers may legally have in their blood. While such laws define legal limits in terms of blood alcohol content (BAC), direct measurement of BAC in a blood specimen is impractical for enforcement purposes. Law enforcement therefore has accepted a correlation between BAC and more simply carried out analyses of alcohol in a person's breath or saliva, and many devices have been developed to analyze a person's breath for alcohol and express the results as BAC.

A six page article, here incorporated by reference in its entirety, by Craig C Freudenreich Ph D entitled “How Breathalyzers Work” is found on the website http://science.howstuffworks.com/breathalyzer.btm. The article reviews the principles of the test methods and the chemical reactions tat take place in each of several different test devices. The following is excerpted from this article:
“Many officers in the field rely on breath alcohol testing devices (Breathalyzer is one type) to determine the blood alcohol concentration (BAC) in drunken-driving suspects.

Alcohol intoxication is legally defined by the blood alcohol concentration (BAC) level. However, taking a blood sample in the field for later analysis in the laboratory is not practical or efficient for detaining drivers suspected of driving while impaired (DWI) or driving under the influence (DUI). Urine tests for alcohol proved to be just as impractical in the field as blood sampling. What was needed was a way to measure something related to SAC without invading a suspect's body.

In the 1940s, breath alcohol testing devices were first developed for use by police. In 1954, Dr. Robert Borkenstein of the Indiana State Police invented the Breathalyzer, one type of breath alcohol testing device used by law enforcement agencies today.

Alcohol that a person drinks shows up in the breath because it gets absorbed from the mouth, throat, stomach and intestines into the bloodstream.

Alcohol is not digested upon absorption, nor chemically changed in the bloodstream. As the blood goes through the lungs, some of the alcohol moves across the membranes of the lung's air sacs (alveoli) into the air, because alcohol will evaporate from a solution—that is, it is volatile. The concentration of the alcohol in the alveolar air is related to the concentration of the alcohol in the blood. As the alcohol in the alveolar air is exhaled, it can be detected by the breath alcohol testing device. Instead of having to draw a driver's blood to test his alcohol level, an officer can test the driver's breath on the spot and instantly know if there is a reason to arrest the driver.

Because the alcohol concentration in the breath is related to that in the blood, you can figure the BAC by measuring alcohol on the breath. The ratio of breath alcohol to blood alcohol is 2,100:1. This means that 2,100 milliliters (ml) of alveolar air will contain the same amount of alcohol as 1 ml of blood.

For many years, the legal standard for drunkenness across the United States was 0.10, but many states have now adopted the 0.08 standard. The federal government has pushed states to lower the legal limit. The American Medical Association says that a person can become impaired when the blood alcohol level hits 0.05. If a person's BAC measures 0.08, it means that there are 0.08 grams of alcohol per 100 ml of blood.

There are three major types of breath alcohol testing devices, and they're based on different principles:
Breathalyzer—Uses a chemical reaction involving alcohol that produces a color change
Intoxilyzer—Detects alcohol by infrared (IR) spectroscopy
Alcosensor III or IV—Detects a chemical reaction of alcohol in a fuel cell

Regardless of the type, each device has a mouthpiece, a tube through which the suspect blows air, and a sample chamber where the air goes. The rest of the device varies with the type.

The Breathalyzer device contains:
A system to sample the breath of the suspect
Two glass vials containing the chemical reaction mixture
A system of photocells connected to a meter to measure the color change associated with the chemical reaction

To measure alcohol, a suspect breathes into the device. The breath sample is bubbled in one vial through a mixture of sulfuric acid, potassium dichromate, silver nitrate and water. The principle of the measurement is based on the following chemical reaction:

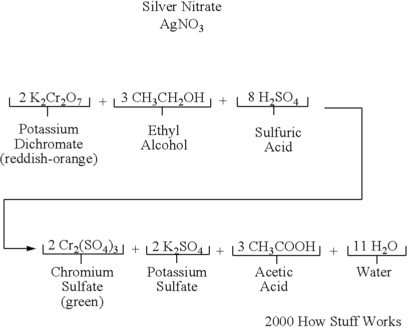

In this reaction:
1. The sulfuric acid removes the alcohol from the air into a liquid solution.
2. The alcohol reacts with potassium dichromate to produce:
a chromium sulfate
potassium sulfate
acetic acid
water

The silver nitrate is a catalyst, a substance that makes a reaction go faster without participating in it. The sulfuric acid, in addition to removing the alcohol from the air, also might provide the acidic condition needed for this reaction.

During this reaction, the reddish-orange dichromate ion changes color to the green chromium ion when it reacts with the alcohol; the degree of the color change is directly related to the level of alcohol in the expelled air. To determine the amount of alcohol in that air, the reacted mixture is compared to a vial of unreacted mixture in the photocell system, which produces an electric current that causes the needle in the meter to move from its resting place. The operator then rotates a knob to bring the needle back to the resting place and reads the level of alcohol from the knob—the more the operator must turn the knob to return it to rest, the greater the level of alcohol.

The Intoxilyzer device uses infrared (IR) spectroscopy, which identifies molecules based on the way they absorb IR light.

In the Intoxilyzer
1. A lamp generates a broadband (multiple-wavelength) IR beam.
2. The broadband IR beam passes through the sample chamber and is focused by a lens onto a spinning filter wheel.
3. The filter wheel contains narrow band filters specific for the wavelengths of the bonds in ethanol. The light passing through each filter is detected by the photocell, where it is converted to an electrical pulse.
4. The electrical pulse is relayed to the microprocessor, which interprets the pulses and calculates the BAC based on the absorption of infrared light.

Modern fuel-cell technology has been applied to breath alcohol detectors. Devices like the Alcosensor III and IV use fuel cells.

The fuel cell has to platinum electrodes with a porous acid-electrolyte material sandwiched between them. As the exhaled air from the suspect flows past one side of the fuel cell, the platinum oxidizes any alcohol in the air to produce acetic acid, protons and electrons.

The electrons flow through a wire from the platinum electrode. The wire is connected to an electrical-current meter and to the platinum electrode on the other side. The protons move through the lower portion of the fuel call and combine with oxygen and the electrons on the other side to font water. The more alcohol that becomes oxidized, the greater the electrical current. A microprocessor measures the electrical current and calculates the BAC.

Operators of any breath alcohol testing device must be trained in the use and calibration of the device, especially if the results are to be used as evidence in DWI trials. Law enforcement officers can carry portable breath testing devices that use Fe same principle as full-size devices. Court cases can turn on the perceived accuracy of a breath test, however, so prosecutors rely on the results obtained from full-size devices.”

It can be observed that the art has greatly improved the ability to understand and quantify the effects of alcohol on people, but the art has apparently learned little or nothing about affecting the elimination of alcohol and its effects beyond letting nature assisted by diminished activity and sleep take its course.

### SUMMARY OF THE INVENTION

In accordance wit this invention, there is provided a method of accelerating the elimination of alcohol from the body and minimizing related ill effects of alcohol intake in a person in need of such treatment, which comprises the administration to such person of an effective amount of at least one compound represented by formula (I) shown below.
X—CH₂—CH(OY)_{q}(H)_{1-q}CH(NH₂⁺)_{1-q}COO⁻ (I)
wherein X represents a trimethylammonio group (CH3)3N+ or a carbamoyl group (CONH₂), Y is hydrogen or an acetyl (CH₃CO) group, and q is zero or one, provided that when X is a carbamoyl group q is zero, and when X is a trimethylammonio group q is one.

Accordingly, in the compound represented by formula (I), when X represents a trimethylammonio group (CH3)3N+ the compound is represented by formula (II)
(CH3)3N+CH₂—CH(OY)CH₂COO⁻ (II)
wherein Y is hydrogen or an acetyl (CH₃CO) group as defined above.

In the compound represented by formula (I), when X represents a carbamoyl group (CONH₂) the compound is represented by formula (III)
(CONH₂)CH₂—CH₂CH(NH₂⁺)COO⁻ (III)

There is also provided, in accordance with this invention, a presentation of a compound represented by formula (I) admixed with edible material in a shaped body characterized by pleasant taste and reduced bulk density compared to the neat compound represented by formula (I) in its conventional powder form.

Elimination of alcohol from the body can occur by exhaling it in the breath, by excreting it in the urine, and by metabolizing it so that it is converted to harmless conversion products such as glycogen, carbon dioxide, and water. Any or all of these processes can be accelerated by administration of a compound or compounds represented by formula (I) according to the invention.

The effectiveness of the compound represented by formula (I) according to the invention is believed to be distinct from the effect of such compound as a nutrient. It is believed to accompany enhancement of the effectiveness of the person's liver in catabolizing alcohol and other toxic compounds to harmless conversion products more rapidly than unassisted.

The present invention is based on the recognition that enhancing the metabolic activity of the liver in a person can be beneficial in augmenting the person's innate ability to exhale, excrete, and metabolize alcohol and increase the rate of clearing alcohol from the liver and the blood stream. This is of considerable practical benefit to consumers of alcohol in enabling such persons to have legal blood levels (i.e. less than 0.08 grams per 100 cc in most states) of alcohol sooner than unassisted after imbibing. Without intending to be bound by any theory, it is believed that the accumulation of alcohol and its metabolic byproducts in the liver and in muscles is slowed as the oxidation of alcohol to harmless conversion products believed to be carbon dioxide and water and possibly the natural energy storage substance glycogen (also known as animal starch) is accelerated. Thus, elevated blood levels of alcohol in a person's blood can be brought down to below legal limits by administration of a compound represented by formula (I) according to this invention. However, administration of at least one compound represented by formula (I) according to this invention also minimizes the undesirable effects that can accompany the consumption of alcohol, such as dizzsiness, nausea, difficulties in walking in a straight line and related diisturbances when the possible consumption is anticipated in the near fuityre. As a result, the quality of life is improved.

In increasing the effectiveness of the human liver according to this invention, doses of 2 to 20 grams of a compound or compounds of formula (I) can be ministered to a person before or after the consumption of alcohol from one to five times a day for a total of 2 to 100 grams per day, preferably 2 to 50 grams per day, in order to accelerate the clearance of alcohol from the person's system.

Such doses can also be administered in advance of or simultaneously with consumption of alcohol. A compound represented by formula (I) can be administered in any convenient manner, as by oral administration in any of the usual dosage forms, such as tablets, capsules, soft gels, solutions and dispersions in liquid foods such as soups and fruit juices. Alternatively, there can be given sterile solutions by direct injection into the bloodstream of the person to be treated, as well as by rectal suppositories. It should be noted that while the net effect of the invention appears to be an improvement in one's ability to “hold one's liquor”, it is not intended to promote an increase in the amount of alcohol consumed by anyone.

In a particularly preferred embodiment, a compound represented by formula (I) can be given in a shaped body characterized by reduced bulk density compared to the neat compound and a pleasant taste and texture welcome in a social situation where alcohol may be consumed, comprising a compound represented by formula (I) and edible material capable of being shaped by heating, blending, pelletizing, rolling, stamping, molding, and other processing techniques consistent with maintaining the chemical integrity of the compound represented by formula (I). It is generally believed that compounds represented by formula (I) are not stable to heat, as such compounds are known to decompose at elevated temperatures without melting. However, it has been found that compounds represented by formula (I) are sufficiently stable to be processed with appropriate edible materials into such products as breadsticks, candies, cookies, doughnuts, gelatin desserts, onion rings, pancakes, potato chips, pretzels, puddings, tempura, tortillas, and wafers, among others. Such shaped bodies typically contain from 10 to 300 parts by weight of compound represented by formula (I) per 100 parts by weight dry basis of edible materials, preferably 20 to 100 parts by weight of compound represented by formula (I) per 100 parts by weight dry basis of edible materials. Suitable edible materials include flour, starch, dextrin, sugar, casein, vegetable protein, gelatin, dried fruit, nuts, butter, vegetable oil, peanut butter and others, as well as appropriate minor amounts of condiments and spices such as salt, pepper, and natural and synthetic flavors,

### EXAMPLE 1

A commercially available instrument was used to analyze breath samples provided by volunteers for alcohol.

TEN volunteers each consumed 5 GLASSES OF WINE on an empty stomach together with a FIFTY GRAM dose of one or more compounds of formula (I) as tabulated below. A control group of volunteers WAS not given any of the compound. The volunteers then provided _EIGHTperiodic breath samples for analysis at intervals of 30 minutes. THE COMPOUNDS GIVEN AND THE SAC LEVELS READ AT EACH SAMPLING ARE TABULATED BELOW

| | | Compound given | | | |
|---|---|---|---|---|---|
| | | | (CONTROL) | (Y = CH3CO) | 2:3 wt |
| | | NONE | II | III | ratioII + III |
| BAC AFTER | 30 MIN. | 0.12 | 0.10 | 0.09 | 0.09 |
| | 60 MIN | 0.15 | 0.09 | 0.08 | .0.08 |
| | 90 MIN | 0.12 | 0.08 | 0.08 | 0.07 |
| | 120 MIN | 0.11 | 0.08 | 0.07 | 0.07 |
| | 150 MIN | 0.11 | 0.07 | 0.07 | 0.06 |
| | 180 MIN | 0.10 | 0.06 | 0.05 | 0.04 |
| | 210 MIN | 0.09 | 0.04 | 0.04 | 0.03 |
| | 240 MIN | 0.08 | 0.03 | 0.03 | 0.02 |

It can be seen from the above results that administration of compound II, III, or their combination helps to diminish the consumer's BAC as measured by the test more rapidly than without such administration. Reduction of the time required for BAC to diminish below 0.08% with FIFTY GRAMS of COMBINED compoundS II AND IN ACCORDANCE WITH THE INVENTION is particularly notable IN LOWERING BAC TO 0.08% IN 60 MINUTES, ONE-FOURTH OF THE TIME REQUIRED WITHOUT THE ASSISTANCE OF THE METHOD OF THE INVENTION.

### EXAMPLE 2

Volunteers were tested to determine a threshold quantity of alcohol (i.e. ounces of vodka given with a slice of bread on an empty stomach) giving rise to symptoms of impairment such as inability to walk 25 feet in a straight line within 30 minutes. One group of volunteers served as a control group. A second group received 20 grams of composition containing several compounds of formula (I) at the same time as the alcohol. A third group received 40 groups of the same composition at the same time.

The average threshold quantity of alcohol for the members of the group receiving 40 grams of composition containing compounds of formula (I) was more than twice the average threshold quantity for the members of the control group.

### EXAMPLE 3

A pancake recipe incorporating a compound represented by formula (I) is prepared as follows:

Flour, 200 grams or approxsimately one standard measuring cup, 50 g rams or ¼ cup sugar, and 125 grams of compound represented by formula (I) are premixed Three eggs are beaten and mixed with one cup of water and one cup of milk. The flour mix is moistened with a little of the egg and milk and then combined with the remaining egg and milk to give a smooth batter.

The batter is baked on a heated and oiled or non-stick skillet or griddle until the top appears no longer drip-wet, about one minute, turned over, baked for an additional minute and remove&

### EXAMPLE 4

A cornstarch pudding recipe incorporating a compound represented by formula (I) is prepared as follows

Sugar (½cup, about 100 grams), ⅓ cup cocoa powder, 3 tablespoons cornstarch and 125 grams of compound represented by formula (I) are premixed, moistened with a small portion of two cups of water (or coffee for mocha flavor) and blended with the remainder of the water. The mixture is heated with stirring until close to boiling and removed from the heat source when it begins to thicken. The hot mixture can be transferred to individual serving size fluted paper molds for cooling and hardening.

### EXAMPLE 5

A gelatin dessert recipe incorporating a compound represented by formula (I) is prepared as follows

The contents of one envelope (about 15 grams) unflavored gelatin, 50 grams sugar, and 100 grams of compound represented by formula (I) are dissolved in one cup (225 ml) of hot water, mixed with one cup of orange juice, and filled into plastic ice cube trays for

1. A method of accelerating the elimination of alcohol in a person in need of such treatment, which comprises the administration to such person of an effective amount of at least one compound represented by formula (I) shown below and inorganic acid addition salts of such compound,
X—CH₂—CH(OY)_{q}(H)_{1-q}CH(NH₂⁺)_{1-q}COO⁻ (I)
wherein X represents a trimethylammonio group (CH3)3N+ or a carbamoyl group (CONH₂), Y is hydrogen or an acetyl (CH₃CO) group, and p and q is zero or one, provided that when X is a carbamoyl group, q is zero, and when X is a trimethylammonio group. q is one.

2. The method of claim 1 wherein the compound is represented by formula (II)
(CH3)3N+CH₂—CH(OY)CH₂COO⁻ (II)
and its inorganic acid addition salts, wherein Y is hydrogen or an acetyl (CH₃CO) group

3. The method of claim 1 wherein the compound is represented by formula (III)
(CONH₂)CH₂—CH₂CH(NH₂⁺)COO⁻ (III)
and its inorganic acid addition salts.

4. The method of claim 1 wherein the compound is administered orally during a period from one hour before alcohol is consumed to one hour after alcohol is consumed.

5. The method of claim 1 wherein the compound is administered orally prior to or simultaneously with consumption of alcohol.

6. The method of claim 1, wherein the compound is administered by rectal suppository.

7. The method of claim 1, wherein the compound is administered in one to five daily doses of 2 to 20 grams each.

8. The method of claim 1, wherein the total of the compound administered daily is in the range of 2 to 100 grams.

9. The method of claim 8, wherein the total of the compound administered daily is in the range of 2 to 50 grams.

10. The method of claim 1 wherein the compound is administered in a single dose of 30 to 60 grams.

11. The method of claim 10, wherein the compound represented by formula (I) is admixed with edible material in a shaped body characterized by pleasant taste and reduced bulk density compared to the neat compound represented by formula (I) in powder form.

12. As an article of manufacture, a shaped body comprising a compound represented by formula (I) and edible material, said body being characterized by pleasant taste and reduced bulk density compared to the neat compound represented by formula (I) in powder form.

13. The article of claim 12, wherein said shaped body is selected from the group consisting of breadsticks, candies, cookies, doughnuts, gelatin desserts, onion rings, pancakes, potato chips, pretzels, puddings, tempura, tortillas, and wafers.

14. The article of claim 12, wherein said shaped body contains from 10 to 300 parts by weight of compound represented by formula (I) per 100 parts by weight dry basis of edible materials.

15. The article of claim 12, wherein said edible materials are selected from the group consisting of flour, starch, dextrin, sugar, casein, vegetable protein, gelatin, dried fruit, nuts, butter, vegetable oil, peanut butter and mixtures thereof, as well as appropriate minor amounts of condiments and spices.
